SEQUENCE LISTING (14 sequences)

SEQ ID NO: 1 - DNA, 1684 bp, Humulus lupulus
actagtttctctccacaatctctgctattgtgcgcactccttcctactcccatgtcgatcactttccacctctgccggatcccaacccgtaccgacctcgccttgccggaggccctctccagaaccggacccttccgctgccgaaaacctttcgttgtccgatgctccggcgagtcatcttccacggcaggagattccgacttcgatgcgaaagtgttccgtaagaacttggttcgaagcaagaactacaatcggaaaggttttggccataaggaagagactcttcagctcatgaacagcgagtacaccagtgatattataaagactttgaaggataacgggaacgagtatagctgggggaacgtgactgtgaaattggccgaagcttatgggttttgctggggtgtggagcgagctgtccagattgcttacgaagcaagaaaacagttccccgaagaaaaaatttggattacaaacgaaatcattcacaatcccacagtcaacaagaggctagaggaaatgaaagtagaaaacattccgattggtgaagggaggaaacaattcgagattgtaaacgagggtgatgttgtgatattgcctgcgtttggtgctggagtggatgagatgttggctttgagtgataggaatgttcaaattgttgataccacatgtccatgggtttccaaggtttggaatacagtcgagaaacataagaaggccgaatacacttccattattcatggtaaatatgctcatgaggagactattgcaactgcatcttttgctggaacttacattattgtaaagaacatgaaagaggcaatgtatgtgtgtgattacattctcggcggtcaactggatggatctagctcaactagagaggagtttatggagaaatttaagaatgctgtttctaagggattcgatcctgacgaacatcttgtgaaggttggtattgcgaatcagaccacaatgctcaagggggaaaccgaagagattgggaaactggttgagagggctatgatgcaaaagtatggagtggaaaacattaatgaacacttccaaagctttaacacaatttgcgatgcgacccaagagcgtcaagatgcaatgtacaagttggtggaggaacctattgatcttatgttagttgttggaggatggaactcgagtaacacctctcatctacaagagattgcagaggaacgtggtattccctcgtattggatcgacagtgaacagagaataggtcctggaaacaaaatagcctacaagctaaatcatggagagttggttgagaaagagaactggttaccagagggtcccatcacgattggtgtaacatcaggtgcttctactccagataaggttgttgaagatgttctcatcaaggtgtttgaccttaaacgcgaagaagctttgcaacttgcttagtttaagttagactcaatgtttgtacatcaccagacagcgctatcatatagcattattattgtatttctaggatgaaatgttgggtccttgttatgcaagagctccatgtatctaagaatattaatcgtcgtatagtagtagaaatcacgttgttattattgaaagggatctaataagttctttgaatctagatagcagccatagcctgcaataatgcgtttaattgctgacacaataatgatattccacc

SEQ ID NO: 2 - DNA, 25 bp, Artificial Sequence
  Description of Artificial Sequence Synthetic primer
actagtttctctccacaatctctgc

SEQ ID NO: 3 - DNA, 25 bp, Artificial Sequence
  Description of Artificial Sequence Synthetic primer
ggtggaatatcattattgtgtcagc

SEQ ID NO: 4 - DNA, 3460 bp, Humulus lupulus
actagtttctctccacaatctctgctattgtgcgcactccttcctactcccatgtcgatcactttccacctctgccggatcccaacccgtaccgacctcgccttgccggaggccctctccagaaccggacccttccgctgccgaaaacctttcgttgtccgatgctccggcgagtcatcttccacggcaggagattccgacttcgatgcgaaagtgttccgtaagaacttggttcgaagcaagaactacaatcggaaaggttttggccataaggaagagactcttcagctcatgaacagcgagtacaccagtatgctgtgcttcgattcctcaatttgtttataatgaatcgtaattatttagtttgattaaataattaatttactggaaaacaaaattcaggtgatattataaagactttgaaggataacgggaacgagtatagctgggggaacgtgactgtgaaattggccgaagcttatgggttttgctggggtgtggagcgagctgtccagattgcttacgaagcaagaaaacagttccccgaagaaaaaatttggattacaaacgaaatcattcacaatcccacagtcaacaaggtttaatcttcattttattttttgattaatgttagtgttttcttcttcgtaatggctcaatttatatatttattaatattattatgattatttggatagaggctagaggaaatgaaagtagaaaacattccgattggtgaagggaggaaacaattcgagattgtaaacgagggtgatgttgtgatattgcctgcgtttggtgctggagtggatgagatgttggctttgagtgataggaatgttcaaattgttgataccacatgtccatgggtttccaaggtaacctttttctatttttatttttttaataaaagatttttaaaatatgggatattgtttaaattatatgttattaatggttttggttgcaaatgctctggtttgaaggtttggaatacagtcgagaaacataagaaggccgaatacacttccattattcatggtaaatatgctcatgaggagactattgcaactgcatcttttgctggaacttacattattgtaaagaacatgaaagaggtaacaactttttctcttatgacaaacaagttttttggtttattctttgtgctgtagattttgatggaatctttattgtcttaaattcaggcagtgtatgtgtgtgattacattctcggcggtcaactggatggatctagctcaactagagaggagtttatggaggtgtgttttgtttattcactttgattaattagttgagaattttcttaaaaacaatttttgaatagttttataatattcgttctttggttttgtctgcagaaatttaagaatgctgtttctaagggattcgatcctgacgaacatcttgtgaaggttggtattgcgaatcagaccacaatgctcaagggggaaaccgaagagattggttagtttttgcaactggttatgctatagatcttgaggaactgccactacttgtttggaacaatgtttaaaatgccgcttttatgcctcaaggggttttgtttttgtgaggcggggcgtacgcctcaaggcgctttgtttttgtgaggcgaggcgtacgcctcaaggcacattgaggcataaacctcaaatatttaattaaaaaaatatttttagaacacctaaagatataaagtaagatgaatgtgtcttataaaactcaaacatagaataaaaaatgtcataaaactcaaacataccataaaatgtaccataaaactcaaacataaatatcttataaattatcaaaattttttaaataaagttaacttttgtttctagaactagaatcctagattccactatcctagaataaacttctgatcagtttttttgggcctatgtatatcagttatgttttaatttgattttggaccttttggacataaaaaacccattacgaaaatacgcccctgaggcgtacgctctctcaatttccacctcagctgccccaactcagcgcctcacctctgctaagcaaggtgcacgccttgttgcgttttttcccacctcaaggcgcacctcaaaaccgcctttttaaacattggtttggaatgtattttgaacttgtattcttaattttcagggaaactggttgaacgccttggtgcgttttttttcgcctcaaggcgcacctcaaaaccgcatttttaaacattggtttggaatgtattttgaacttgtattcttaattttcagggaaactggttgagagggctatgatgcaaaagtatggagtggaaaacattaatgaacacttccaaagctttaacacaatttgcgatgcgacccaagtaataagtcccttcctcttctgtacatgtatccttgtctggtttgttatttctgagatttcatttcactcaaccgaaatccttttcttttccttgtttgctcatataatagattgacgacattcctcttgtgtcatgtagtctgaactaattttttatgttttgtcgtttcccaggagcgtcaagatgcaatgtacaagttggtggaggaacctattgatcttatgttagttgttggaggatggaactcgagtaacacctctcatctacaagagattgcagagaaacgtggtattccctcatattggatcgacagtgaacagagaataggtcctggaaacaaaatagcctacaagctaaatgtaagtttaaacagattaggctaccattacaatctatttatttctggttttctgctaatgtactagtttagcagaactccttagcaatgttttcttcttatctgtggtcctgattgttgccaatttcttcttatgaattttctgatagcatggagagttggttgagaaagagaactggttaccagagggtcccatcacgattggtgtaacatcaggtgcttctactccagataaggtgagttcttattcacaaaatccttaaaaacagcaaaccacctcaatgaaatcatgaaaagccatgataaaccatattcttcattctactcataagacacaaggaagtgcaaatttttctcattgattttttatggttgtacatctctctgctattttctgaatcaggttgttgaagatgttctcatcaaggtgtttgaccttaaacgcgaagaagctttgcaacttgcttagtttaagttagactcaatgtttgtacatcaccagacagcgctatcatatagcattattattgtatttctaggatgaaatgttgggtccttgttatgcaagagctccatgtatctaagaatattaatcgtcgtatagtagtagaaatcacgttgttattattgaaagtgatctaataagttctttgaatctagatagcagccatagcctgcaataatgcgtttaattgctgacacaataatgatattccacc

SEQ ID NO: 5 - DNA, 3434 bp, Humulus lupulus
actagtttctctccacaatctctgctattgtgcgcactccttcctactcccatgtcgatcactttccacctctgccggatcccaacccgtaccgacctcgccttgccggaggccctctccagaaccggacccttccgctgccgaaaacctttcgttgtccgatgctccggcgagtcatcttccacggcaggagattccgacttcgatgcgaaagtgttccgtaagaacttggttcgaagcaagaactacaatcggaaaggttttggccataaggaagagactcttcagctcatgaacagcgagtacaccagtatgctgtgcttcgattcctcaatttgtttataatgaatcgtaattatttagtttgattaaataattaatttactggaaaacaaaattcaggtgatattataaagactttgaaggataacgggaacgagtatagctgggggaacgtgactgtgaaattggccgaagcttatgggttttgctggggtgtggagcgagctgtccagattgcttacgaagcaagaaaacagttccccgaagaaaaaatttggattacaaacgaaatcattcacaatcccacagtcaacaaggtttaatcttcattttattttttaattaatgttagtgttttcttcttcgtaatggctcaatttatatatttattaatattattatgattatttggatagaggctagaggaaatgaaagtagaaaacattccgattggtgaagggaggaaacaattcgagattgtaaacgagggtgatgttgtgatattgcctgcgtttggtgctggagtggatgagatgttggctttgagtgataggaatgttcaaattgttgataccacatgtccatgggtttccaaggtaacctttttctatttttatttttttaataaaagatttttaaaatatgggatattgtttattatatgttattaatggttttgtttgcaaatgctctggtttgaaggtttggaatacagtcgagaaacataagaaggccgaatacacttccattattcatggtaaatatgctcatgaggagactattgcaactgcatcttttgctggaacttacattattgtaaagaacatgaaagaggtaacaactttttctcttatgacaaacaagttttttggtttattctttgtgctgtagattttgatggaatctttattgtcttaaattcaggcaatgtatgtgtgtgattacattctcggcggtcaactggatggatctagctcaactagagaggagtttatggaggtgtgttttgtttattcactttgattaattagttgacaattttcttaaaaacaatttttgaatagttttataatattcgttctttggttttgtctgcagaaatttaagaatgctgtttctaagggattcgatcctgacgaacatcttgtgaaggttggtattgcgaatcagaccacaatgctcaagggggaaaccgaagagattggttagtttttgcaactggttatgctatagatcttgaggaactgccactacttgtttggaacaatgtttaaaatgccgcttttatgcctcaaggggttttgtttttgtgaggcgaggcgtacgcctcaaggcacattgaggcataaacctcaaatatttaattaaaaaaatatttttagaacacctaaagatataaagtaagatgaatgtgtcctataaaactcaaacatagaataaaaaatgtcataaaactcaaacataccataaaatgtaaatgtaccataaaactcaaacataaatatcttataaattatctaaattttttaaataaagtaacttttgtttctagaactagaatcctagattccactatcctagaataaacttctgatcagtttttttgggcctatttatatcagttgtgttttaatttgatttggaccttttggacataaaaaacccattacgaaaatacgcccctgaggcgtacgctctctcaatttccacctcagctgtcccaactcagcgcctcactacagtgaggtgtatgcctcacctctgctaagcaaggtgcacgccttggtgcgtttttttccacctcaaggcgcacctcaaaactgcctttttaaacattggtttggaatgtattttgaacttgtattcttaattttcagggaaactggttgaacgccttggtgcgtttttttgcgcctcaaggcacacatcaaaaccgcatttttaaacattggtttggaatgtattcttaattttcagggaaactggttgagagggctatgatgcaaaagtatggagtggaaaacattaatgaacacttccaaagctttaacacaatttgcgatgcgacccaagtaataagtcccttcctcttctgtacatgtatccttgtctggtttgttatttctgagatttcatttcactcaaccgaaatccttttcttttccttgtttgctcatataatagattgacgacattcctcttgtgtcatgtagtctgaactaattttttatgttttgtcgtttcccaggagcgtcaagatgcaatgtacaagttggtggaggaacctattgatcttatgttagttgttggaggatggaactcgagtaacacctctcatctacaagagattgcagaggaacgtggtattccctcgtattggatcgacagtgaacagagaataggtcctggaaacaaaatagcctacaagctaaatgtaagtttaaacagattaggctaccattacaatctatttatttctggttttctgtcaatgtactagtttagcagaactccttagcaatgttttcttcttatctgtggtcctgattgttgtcaatttcttcttatgaattttctgatagcatggagagttggttgagaaagagaactggttaccagagggtcccatcacgattggtgtaacatcaggtgcttctactccagataaggtgagttcttattcacaaaatccttaaaaacagcaaaccacttcaatgaaatcatgaaaagccatgataaaccatattcttcattctactcataagacacaaggaagtgcaaatttttctcattgattttttatggttgtacatctctctgctattttctgaatcaggttgttgaagatgttctcatcaaggtgtttgaccttaaacgcgaagaagctttgcaacttgcttagtttaagttagactcaatgtttgtacatcaccagacagcgctatcatatagcattattattgtatttctaggatgaaatgttgggtccttgttatgcaagagctccatgtatctaagaatattaatcgtcgtatagtagtagaaatcacgttgttattattgaaagggatctaataagttctttgaatctagatagcagccatagcctgcaataatgcgtttaattgctgacacaataatgatattccacc

SEQ ID NO: 6 - DNA, 3460 bp, Humulus lupulus
actagtttctctccacaatctctgctattgtgcgcactccttcctactcccatgtcgatcactttccacctctgccggatcccaacccgtaccgacctcgccttgccggaggccctctccagaaccggacccttccgctgccgaaaacctttcgttgtccgatgctccggcgagtcatcttccacggcaggagattccgacttcgatgcgaaagtgttccgcaagaacttggttcgaagcaagaactacaatcggaaaggttttggccataaggaagagactcttcagctcatgaacagcgagtacaccagtatgctgtgcttcgattcctcaatttgtttataatgaatcgtaattatttagtttgattaaataattaatttactggaaaacaaaattcaggtgatattataaagactttgaaggataacgggaacgagtatagctgggggaacgtgactgtgaaattggccgaagcttatgggttttgctggggtgtggagcgagctgtccagattgcttacgaagcaagaaaacagttccccgaagaaaaaatttggattacaaacgaaatcattcacaatcccacagtcaacaaggtttaatcttcattttattttttgattaatgttagtgttttcttcttcgtaatggctcaatttatatatttattaatattattatgattatttggatagaggctagaggaaatgaaagtagaaaacattccgattggtgaagggaggaaacaattcgagattgtaaacgagggtgatgttgtgatattgcctgcgtttggtgctggagtggatgagatgttggctttgagtgataggaatgttcaaattgttgataccacatgtccatgggtttccaaggtaacctttttctatttttatttttttaataaaagatttttaaaatatgggatattgtttaaattatatgttattaatggttttggttgcaaatgctctggtttgaaggtttggaatacagtcgagaaacataagaaggccgaatacacttccattattcatggtaaatatgctcatgaggagactattgcaactgcatcttttgctggaacttacattattgtaaagaacatgaaagaggtaacaactttttctcttatgacaaacaagttttttggtttattctttgtgctgtagattttgatggaatctttattgtcttaaattcaggcagtgtatgtgtgtgattacattctcggcggtcaactggatggatctagctcaactagagaggagtttatggaggtgtgttttgtttattcactttgattaattagttgagaattttcttaaaaacaatttttgaatagttttataatattcgttctttggttttgtctgcagaaatttaagaatgctgtttctaagggattcgatcctgacgaacatcttgtgaaggttggtattgcgaatcagaccacaatgctcaagggggaaaccgaagagattggttagtttttgcaactggttatgctatagatcttgaggaactgccactacttgtttggaacaatgtttaaaatgccgcttttatgcctcaaggggttttgtttttgtgaggcggggcgtacgcctcaaggcgctttgtttttgtgaggcgaggcgtacgcctcaaggcacattgaggcataaacctcaaatatttaattaaaaaaatatttttagaacacctaaagatataaagtaagatgaatgtgtcttataaaactcaaacatagaataaaaaatgtcataaaactcaaacataccataaaatgtaccataaaactcaaacataaatatcttataaattatcaaaattttttaaataaagttaacttttgtttctagaactacaatcctagattccactatcctagaataaacttctgatcagtttttttgggcctatgtatatcagttatgttttaatttgattttggaccttttggacataaaaaacccattacgaaaatacgcccctgaggcgtacgctctctcaatttccacctcagctgccccaactcagcgcctcacctctgctaagcaaggtgcacgccttgttgcgttttttcccacctcaaggcgcacctcaaaaccgcctttttaaacattggtttggaatgtattttgaacttgtattcttaattttcagggaaactggttgaacgccttggtgcgttttttttcgcctcaaggcgcacctcaaaaccgcatttttaaacattggtttggaatgtattttgaacttgtattcttaattttcagggaaactggttgagagggctatgatgcaaaagtatggagtggaaaacattaatgaacacttccaaagctttaacacaatttgcgatgcgacccaagtaataagtcccttcctcttctgtacatgtatccttgtctggtttgttatttctgagatttcatttcactcaaccgaaatccttttcttttccttgtttgctcatataatagattgacgacattcctcttgtgtcatgtagtctgaactaattttttatgttttgtcgtttcccaggagcgtcaagatgcaatgtacaagttggtggaggaacctattgatcttatgttagttgttggaggatggaactcgagtaacacctctcatctacaagagattgcagaggaacgtggtattccctcatattggatcgacagtgaacagagaataggtcctggaaacaaaatagcctacaagctaaatgtaagtttaaacagattaggctaccattacaatctatttatttctggttttctgctaatgtactagtttagcagaactccttagcaatgttttcttcttatctgtggtcctgattgttgccaatttcttcttatgaattttctgatagcatggagagttggttgagaaagagaactggttaccagagggtcccatcacgattggtgtaacatcaggtgcttctactccagataaggtgagttcttattcacaaaatccttaaaaacagcaaaccacctcaatgaaatcatgaaaagccatgataaaccatattcttcattctactcataagacacaaggaagtgcaaatttttctcattgattttttatggttgtacatctctctgctattttctgaatcaggttgttgaagatgttctcatcaaggtgtttgaccttaaacgcgaagaagctttgcaacttgcttagtttaagttagactcaatgtttgtacatcaccagacagcgctatcatatagcattattattgtatttctaggatgaaatgttgggtccttgttatgcaagagctccatgtatctaagaatattaatcgtcgtatagtagtagaaatcacgttgttattattgaaagtgatctaataagttctttgaatctagatagcagccatagcctgcaataatgcgtttaattgctgacacaataatgatattccacc

SEQ ID NO: 7 - DNA, 20 bp, Artificial Sequence
  Description of Artificial Sequence Synthetic primer
tgctgtttctaagggattcg

SEQ ID NO: 8 - DNA, 20 bp, Artificial Sequence
  Description of Artificial Sequence Synthetic primer
tttccaggacctattctctg

SEQ ID NO: 9 - DNA, 30 bp, Artificial Sequence
  Description of Artificial Sequence Synthetic oligonucleotide
tttttttttttttttttttttttttttttt

SEQ ID NO: 10 - DNA, 120 bp, Humulus lupulus
tttaatcttcattttattttttgattaatgttagtgttttcttcttcgtaatggctcaatttatatatttattaatattattatgattatttggatagaggctagaggaaatgaaagtag

SEQ ID NO: 11 - DNA, 120 bp, Humulus lupulus
tttaatcttcattttattttttaattaatgttagtgttttcttcttcgtaatggctcaatttatatatttattaatattattatgattatttggatagaggctagaggaaatgaaagtag

SEQ ID NO: 12 - DNA, 120 bp, Humulus lupulus
gtaccataaaactcaaacataaatatcttataaattatcaaaattttttaaataaagttaacttttgtttctagaactagaatcctagattccactatcctagaataaacttctgatcag

SEQ ID NO: 13 - DNA, 119 bp, Humulus lupulus
gtaccataaaactcaaacataaatatcttataaattatctaaattttttaaataaagtaacttttgtttctagaactagaatcctagattccactatcctagaataaacttctgatcag

SEQ ID NO: 14 - DNA, 120 bp, Humulus lupulus
gtaccataaaactcaaacataaatatcttataaattatcaaaattttttaaataaagttaacttttgtttctagaactacaatcctagattccactatcctagaataaacttctgatcag

(19) United States
(12) Patent Application Publication
(10) Pub. No.: US 2010/0279284 A1
(43) Pub. Date: Nov. 4, 2010

(54) METHOD FOR SELECTING HOP LINE AND BREEDING MARKER AND PRIMER SET USED FOR SELECTING HOP LINE

(75) Inventors: Yukio Okada, Tokyo (JP); Koichiro Koie, Tokyo (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: SAPPORO BREWERIES LIMITED, Shibuya-ku (JP)

(21) Appl. No.: 12/447,987

(22) PCT Filed: Oct. 29, 2007
(86) PCT No.: PCT/JP2007/071021

§ 371 Date: Jun. 8, 2009

(30) Foreign Application Priority Data
Oct. 30, 2006 (JP) 2006-294798

(51) Int. Cl. C12Q 1/68; C07H 21/04

(52) U.S. Cl. 435/6; 536/23.1

(57) ABSTRACT

It is an object of the invention to screen for hop varieties with low cohumulone ratio and low colupulone ratio, as well as hop varieties with high contents of farnesene and/or linalool within a short time period utilizing a molecular screening method that employs a breeding marker. The invention provides a breeding marker using a polymorphism at the position of the 623rd nucleotide or a polymorphism at the position of the 1820th nucleotide of a polynucleotide consisting of the nucleotide sequence as set forth in SEQ ID NO: 5.

### TECHNICAL FIELD

The present invention relates to a method for screening of hop varieties and to a breeding marker and primer set to be used for screening of hop varieties.

### BACKGROUND ART

Hops, the primary material for beer, impart a refreshing bitterness and aroma to beer. The known bittering agents in hops include α acids such as humulone, cohumulone and adhumulone, and β acids such as lupulone, colupulone and adlupulone. The α acids exhibit antibacterial activity, anticancer activity, anti-inflammatory effects and so on, and the β acids exhibit sedative effects and so on, and thus, they are both being considered also as drug sources (Non-patent document 1).

The α acid contained in hops, when it is treated with heat during a beer production process, is isomerized into iso-α acid (iso-humulone, iso-cohumulone and iso-adhumulone). It is known that when iso-humulone and iso-cohumulone (which occupy a major part of iso α acids) are compared, iso-cohumulone is inferior in sensuality, more specifically, the quality of bitterness is low, and also inferior in beer-foam stabilization (Non-Patent Document 2). Since iso-cohumulone is produced by isomerization of cohumulone, it is desired to screen for hop varieties having a low ratio of the cohumulone contents (herein after, cohumulone ratio) relative to the contents of the α acid contents in hops, in breeding hops.

In the β-acids contained in hops, lupulone and colupulone are mostly included. It is known that degree of bitterness can be reduced (milder) or increased (stronger) depending upon the contents of β-acids in hops (Non-Patent Document 3).

It is known that farnesene is a component detected only in the top-grade hops called fine aroma hop, whereas linalool imparts rose-like flower scent to beer (Non-Patent Document 2). Farnesene and linalool contained in hops are considered as an important component for characterizing beer in breeding hops.

Methods used for breeding of useful plants include methods in which high-grade varieties are crossed and the obtained hybrid progeny are cultivated until expression of the desired traits and screened, and molecular screening methods in which the genomic DNA of hybrid progeny are examined at the seed or seedling stage and screened based on breeding markers (DNA markers).

[Non-Patent Document 1] Gerhaeuser, European Journal of Cancer, 2005, Vol. 41, No. 13, p. 1941-1954
[Non-Patent Document 2] Peacock et al., European Brewing Convention monograph XXII Symposium on hops Zoeterwoude, 1994, p. 247-250
[Non-Patent Document 3] Kowaka et al., EBC proceedings, 1983, Vol. 19, p. 71-78
[Non-Patent Document 4] Peacock et al., J. Agric. Food Chem., 1980, Vol. 28, p. 774-777

### DISCLOSURE OF THE INVENTION

### Problems to be Solved by the Invention

However, virtually no genetic research has been conducted on male hop plants, which produce no cones, and virtually none of the genes involved in biosynthesis of the aforementioned components associated with hop bitterness and aroma have been identified even in female plants. Consequently, it has not been possible to utilize molecular screening methods based on breeding markers, for screening of hop varieties based on ratio of cohumulone and/or colupulone and contents of farnesene and/or linalool.

In addition, since α acids, β acids, farnesene and linalool accumulate specifically in the lupulin gland of hop cones, screening of hop varieties with high levels of these components can only be done after seeding and waiting for forming of the cones, and at least several years of cultivation have been necessary for stabilization of these traits.

It is therefore an object of the present invention to utilize a breeding marker-based molecular screening method for screening of hop varieties with low cohumulone ratio, within a shorter time period. It is another object of the invention to screen for hop varieties with low ratio of cohumulone and colupulone, and with high contents of farnesene and/or linalool, within a shorter time period.

### Means for Solving the Problems

In order to achieve the objects stated above, the invention provides breeding markers, represented by any one of the following (a) to (d), to be used for screening of hop varieties with low ratio of cohumulone contents relative to α acid contents (cohumulone ratio) in hops.
(a) A polynucleotide consisting of a nucleotide sequence of 20-3434 continuous nucleotides including nucleotide No.623 of the polynucleotide consisting of the nucleotide sequence as set forth in SEQ ID NO: 5 (wherein the 623th nucleotide has adenine).
(b) A polynucleotide consisting of the sequence complementary to the polynucleotide of (a) above.
(c) A polynucleotide consisting of a nucleotide sequence of 20-3434 continuous nucleotides including nucleotide No.1820 of the polynucleotide consisting of the nucleotide sequence as set forth in SEQ ID NO: 5 (wherein the 1820th nucleotide has adenine).
(d) A polynucleotide consisting of the sequence complementary to the polynucleotide of (c) above.

The present inventors have discovered the 623rd nucleotide (having adenine) and the 1820th nucleotide (having adenine) of the polynucleotide consisting of the nucleotide sequence as set forth in SEQ ID NO: 5, as a polymorphism found to be common to hop varieties with low cohumulone ratio, from a cDNA library prepared from the lupulin gland of hop cones. No breeding marker for anticipation of low cohumulone ratio that can be applied to all hops has been reported to date, but it was found that this breeding marker allows screening of hop varieties with low cohumulone ratio among hybrid progeny obtained by crossing various hop lines. Also, it was demonstrated that the hop varieties screened with the breeding marker also have low colupulone ratio and high contents of farnesene and/or linalool.

If, during breeding of hop varieties, those hop varieties having the same sequence as the breeding marker are selected, it is possible to screen for hop varieties with low cohumulone ratio at the seed or seedling stage without waiting for the hops to grow and form cones. Moreover, determining the presence of the breeding marker allows objective, genomic information-based identification of the combinations of crossed lines, that has hitherto depended on intuition and experience. The breeding marker may also be suitably used for screening of hop varieties with low colupulone ratio and with high contents of farnesene and/or linalool.

Here, the phrase “hop varieties with low cohumulone ratio” refers to hop varieties in groups with low average values for cohumulone ratio, when groups of hybrid progeny are categorized according to whether or not they have the same sequence as the aforementioned breeding marker. The phrase “hop varieties with low colupulone ratio” refers to hop varieties in groups with low average values for colupulone ratio, when groups of hybrid progeny are categorized according to whether or not they have the same sequence as the aforementioned breeding marker. The phrase “hop varieties with high contents of farnesene and/or linalool” refers to hop varieties in groups with high average values for contents of farnesene and/or linalool, when groups of hybrid progeny are categorized according to whether or not they have the same sequence as the aforementioned breeding marker.

The invention still further provides a screening method for hop varieties with low ratio of cohumulone contents relative to α acid contents (low cohumulone ratio) in hops, comprising: an extraction step in which genomic DNA is extracted from a hop variety specimen; a digestion step in which the genomic DNA is digested with restriction enzyme XbaI to obtain digested genomic DNA fragments; a detection step in which the digested genomic DNA fragments are separated and genomic DNA fragments that hybridize to any one of the polynucleotide of (a) to (d) above are detected; And a judging step in which it is judged that the variety has low cohumulone ratio when a genomic DNA fragment of about 8.6 kbp is not detected in the detection step above.

With this screening method it is possible to screen for hop varieties with low cohumulone ratio, hop varieties with low colupulone ratio, and hop varieties with high contents of farnesene and/or linalool, by using any one of the polynucleotide of (a) to (d) as a probe and examining Restriction Fragment Length Polymorphisms (RFLP) with restriction enzyme XbaI. Since genome fragment sizes can be visually discriminated, it is possible to objectively and rapidly screen for hop varieties having these features without examining the nucleotide sequences of the genomic DNA.

The invention yet further provides a method for screening of hop varieties with low ratio of cohumulone contents relative to α acid contents (low cohumulone ratio) in hops, which comprises an extraction step in which genomic DNA is extracted from a hop variety specimen, and an identifying step in which genomic DNA polymorphism at the 623rd nucleotide position of the nucleotide sequence as set forth in SEQ ID NO: 5 is identified.

The identifying step preferably comprises a PCR step in which DNA is synthesized by PCR using the aforementioned genomic DNA as template, a primer consisting of a nucleotide sequence of 18-50 contiguous nucleotides located toward the 5′-end from the 623rd nucleotide of the nucleotide sequence as set forth in SEQ ID NO: 5 and a primer consisting of the sequence complementary to a nucleotide sequence of 18-50 contiguous nucleotides located toward the 3′-end from the 623rd nucleotide of the nucleotide sequence as set forth in SEQ ID NO: 5, a digestion step in which the DNA is digested with restriction enzyme PacI, and a judging step in which the variety is judged to have low cohumulone ratio when the DNA is not cleaved with restriction enzyme PacI in the digestion step above.

In this screening method, PCR is performed using the extracted genomic DNA as a template and whether or not the amplified DNA is cleaved with restriction enzyme PacI is used as an index. Therefore no Southern hybridization is necessary. If the sizes of the DNA fragments after the digestion step are confirmed based on, for example, fractionation by agarose electrophoresis, whether or not the synthesized DNA in the PCR step was cleaved by restriction enzyme PacI can be easily determined, thus allowing screening of hop varieties with low cohumulone ratio or hop varieties with low colupulone ratio as well as low cohumulone ratio, and hop varieties with high contents of farnesene and/or linalool.

The present invention also provides a method for screening of hop varieties with low ratio of cohumulone contents relative to α acid contents (cohumulone ratio) in hops which comprises: an extraction step in which genomic DNA is extracted from a hop variety specimen; and an identifying step in which genomic DNA polymorphism at the 1820th nucleotide position of the nucleotide sequence as set forth in SEQ ID NO: 5 is identified.

It is preferred that the identifying step comprises: a PCR step in which DNA is synthesized by PCR using the aforementioned genomic DNA as template, a primer consisting of a nucleotide sequence of 18-50 contiguous nucleotides located toward the 5′-end from the 1820th nucleotide of the nucleotide sequence as set forth in SEQ ID NO: 5 and a primer consisting of the sequence complementary to a nucleotide sequence of 18-50 contiguous nucleotides located toward the 3′-end from the 1820th nucleotide of the nucleotide sequence as set forth in SEQ ID NO: 5; a digestion step in which the DNA is digested with restriction enzyme HpaI; and a judging step in which the variety is judged to have low cohumulone ratio when all kinds of the DNA are cleaved with restriction enzyme HpaI in the digestion step above.

In this screening method, PCR is performed using the extracted genomic DNA as a template and whether or not the amplified DNA is cleaved with restriction enzyme HpaI is used as an index. Therefore no Southern hybridization is necessary. If the sizes of the DNA fragments after the digestion step are confirmed based on, for example, fractionation by agarose electrophoresis, whether or not the synthesized DNA in the PCR step was cleaved by restriction enzyme HpaI can be easily determined, thus allowing screening of hop varieties with low cohumulone ratio or hop varieties with low colupulone ratio as well as low cohumulone ratio, and hop varieties with high contents of farnesene and/or linalool.

It is preferred that the identifying step comprises: a PCR step in which DNA is synthesized by PCR using the aforementioned genomic DNA as template, a primer including the nucleotide sequence as set forth in SEQ ID NO: 7 and a primer including the nucleotide sequence as set forth in SEQ ID NO: 8; a digestion step in which the DNA is digested with restriction enzyme HpaI; and a judging step in which the variety is judged to have low cohumulone ratio when a DNA fragment of about 1304 by is not contained in the DNA fragments obtained by the digestion step above.

In this screening method, since PCR is conducted using the extracted genomic DNA as template, a primer containing the nucleotide sequence as set forth in SEQ ID NO: 7 and a primer containing the nucleotide sequence as set forth in SEQ ID NO: 8, only the target DNA fragment is efficiently and specifically amplified. In addition, if the sizes of the DNA fragments obtained by the digestion step are confirmed based on, for example, fractionation by agarose electrophoresis, whether or not an approximately 1304 by fragment is present can be easily determined, thus allowing screening of hop varieties with low cohumulone ratio or hop varieties with low colupulone ratio as well as low cohumulone ratio, and hop varieties with high contents of farnesene and/or linalool.

The present invention also provides a primer set to be used for screening of hop varieties with low ratio of cohumulone contents relative to α acid contents (cohumulone ratio)in hops, the primer set comprising: a primer containing the nucleotide sequence as set forth in SEQ ID NO: 7; And a primer containing the nucleotide sequence as set forth in SEQ ID NO: 8.

Using this primer set allows specific amplification of only the DNA fragment containing the 1820th nucleotide of the nucleotide sequence as set forth in SEQ ID NO: 5, using the extracted genomic DNA as template. The primer set can be appropriately utilized for screening of hop varieties with low cohumulone ratio or hop varieties with low colupulone ratio as well as low cohumulone ratio, and hop varieties with high contents of farnesene and/or linalool.

### Effect of the Invention

According to the invention it is possible to provide a breeding marker for hop varieties with low cohumulone ratio or hop varieties with low colupulone ratio as well as low cohumulone ratio, and hop varieties with high contents of farnesene and/or linalool. According to the invention it is also possible to objectively and rapidly screen for hop varieties with low cohumulone ratio or hop varieties with low colupulone ratio as well as low cohumulone ratio, and hop varieties with high contents of farnesene and/or linalool, using hop seeds or seedlings, without waiting for the hops to form cones and develop stabilized traits.

### BRIEF DESCRIPTION OF THE DRAWINGS

FIG. 1 is an image showing XbaI/3D03 polymorphism, obtained by digestion of genomic DNA of hop varieties with restriction enzyme XbaI, and genomic Southern hybridization.

FIG. 2 shows a single nucleotide polymorphism obtained by analysis of homology of the nucleotide sequences of e-type g3D03, f-type g3D03 and g-type g3D03m.

FIG. 3 shows a typical band pattern for HpaI/g3D03 polymorphism, obtained by PCR-RFLP of hop varieties.

FIG. 4 is an image showing HpaI/g3D03 polymorphism, obtained by analysis of genomic DNA of 9 hop lines.

FIG. 5 is an image showing XbaI/3D03 polymorphism, obtained by genomic analysis of 9 hop lines.

### BEST MODES FOR CARRYING OUT THE INVENTION

Preferred embodiments of the invention will now be described in detail.

The breeding markers of the invention are characterized by being represented by any one of the following (a) to (d), which is used for screening of hop varieties with low ratio of cohumulone contents relative to α acid contents (cohumulone ratio) in hops.
(a) A polynucleotide consisting of a nucleotide sequence of 20-3434 continuous nucleotides including nucleotide No.623 of the polynucleotide consisting of the nucleotide sequence as set forth in SEQ ID NO: 5 (wherein the 623th nucleotide has adenine).
(b) A polynucleotide consisting of the sequence complementary to the polynucleotide of (a) above.
(c) A polynucleotide consisting of a nucleotide sequence of 20-3434 continuous nucleotides including nucleotide No.1820 of the polynucleotide consisting of the nucleotide sequence as set forth in SEQ ID NO: 5 (wherein the 1820th nucleotide has adenine).
(d) A polynucleotide consisting of the sequence complementary to the polynucleotide of (c) above.

The term “breeding marker” means a DNA marker used for breeding, and it is a DNA sequence that exists in a genome near a gene associated with a trait of interest and operates together with the gene. A breeding marker allows identification of traits that cannot be distinguished by the naked eye, such as disease resistance, or traits that appear in the mature phase, by using genomic DNA from seeds or seedlings of the plant specimen. The breeding marker may be the gene itself, or DNA other than the gene.

The term “cohumulone ratio” means the ratio of cohumulone contents relative to the contents of α acid in hops. The term “colupulone ratio” means the ratio of the colupulone contents relative to the contents of β acid in hops. The term “α acid” refers to a bitter substance in hops that is primarily humulone and its analogs including cohumulone, adhumulone and so on. The term “β acid” refers to a bitter substance in hops that is primarily lupulone and its analogs including colupulone, adlupulone and so on.

Throughout the present specification, “hops” refers to the perennial plant (Humulus lupulus L.) itself, and “cones” refers to the female flower produced by female hops, where the perichaetium has formed a pine cone-shaped chamber. The term “hop variety” means each hybrid progeny obtained by breeding hops.

The term “polynucleotide” refers to a molecule with a plurality of bonded phosphoric acid esters of nucleosides comprising purine or pyrimidine bases that are β-N-glycoside bonded to sugars (ribonucleotides or deoxyribonucleotides).

The polynucleotides represented by (a) to (d) above may be cloned from a hop genomic library using a labeled DNA probe synthesized based on the nucleotide sequence information of SEQ ID NO: 5, according to the method described in, for example, Molecular cloning (Maniatis et al., 1989, Cold Spring Harbor Laboratory Press).

The polynucleotides represented by (a) to (d) may also be synthesized by PCR using a primer set designed based on the nucleotide sequence information of SEQ ID NO: 5, with genomic DNA extracted from tissue of hop leaves or the like as template. Also, partial sequences of the polynucleotide represented by (a) to (d) may be synthesized with a DNA synthesizer and linked by an enzymatic method and subcloning, to obtain a polynucleotide of the target size.

The breeding marker may be used for screening of hop varieties with low cohumulone ratio, but it is preferably used for screening of hop varieties with low colupulone ratio as well as low cohumulone ratio, and hop varieties with high contents of farnesene and/or linalool.

Also, the screening method according to the invention, which is for hop varieties with low ratio of cohumulone contents relative to α acid contents in hops, is characterized by comprising: an extraction step in which genomic DNA is extracted from a hop variety specimen; a digestion step in which the genomic DNA is digested with restriction enzyme XbaI to obtain digested genomic DNA fragments; a detection step in which the digested genomic DNA fragments are separated and genomic DNA fragments that hybridize to any one of the polynucleotide of (a) to (d) above are detected; and a judging step in which it is judged that the variety has low cohumulone ratio when a genomic DNA fragment of about 8.6 kbp is not detected in the detection step above.

Methods utilizing RFLP may be mentioned as examples of such screening methods. Specifically, this method compares the sizes of DNA fragments produced after treatment with a restriction enzyme, based on mutations or nucleotide insertions or deletions at a restriction enzyme recognition site in the genomic DNA, to determine whether or not a certain trait is present.

In the detection step, it is possible to detect genomic DNA fragments that hybridize to the polynucleotides represented by (a) to (d) by genomic Southern hybridization, as described in Molecular cloning (Maniatis et al., 1989, Cold Spring Harbor Laboratory Press), for example. Specifically, the digested genomic DNA fragments of a hop specimen digested with restriction enzyme XbaI are fractionated by agarose gel electrophoresis, the fractionated genomic DNA fragments are transferred to a nylon membrane, a labeled polynucleotide represented by (a) to (d) above is used as a probe for hybridization, and the hybridized bands are detected based on the probe labeling.

The probe labeling method may be a method in which a DNA polymerase such as Klenow enzyme is used to incorporate substrate nucleotides that are labeled with an isotope such as ³²P, a fluorescent dye, digoxigenin (DIG) or biotin, using random hexamer oligonucleotides and the like as primer (random primer method), or a method in which T4 polynucleotide kinase is used for phosphorylation of the 5′-end of the oligonucleotide with ³²P or the like for labeling.

In the judging step, digested genomic DNA fragments from a hop variety specimen may be electrophoresed with a DNA size marker and transferred to a nylon membrane, with the well positions and size marker migration positions marked on the nylon membrane, thus allowing the sizes of the digested genomic DNA fragments from hybridized hop variety specimens to be calculated based on the relationship between migration distance from the well and DNA size marker size. As a result, if a digested genomic DNA fragment of approximately 8.6 kbp is not detected, the hop variety specimen may be judged to be a variety with low cohumulone ratio.

Also, the screening method according to the invention, which is for hop varieties with low ratio of cohumulone contents relative to a acid contents in hops, is characterized by comprising: an extraction step in which genomic DNA is extracted from a hop variety specimen; and an identifying step in which genomic DNA polymorphism at the 623rd nucleotide position of the nucleotide sequence as set forth in SEQ ID NO: 5 is identified.

The identifying step preferably comprises: a PCR step in which DNA is synthesized by PCR using the aforementioned genomic DNA as template, a primer consisting of a nucleotide sequence of 18-50 contiguous nucleotides located toward the 5′-end from the 623rd nucleotide of the nucleotide sequence as set forth in SEQ ID NO: 5 and a primer consisting of the sequence complementary to a nucleotide sequence of 18-50 contiguous nucleotides located toward the 3′-end from the 623rd nucleotide of the nucleotide sequence as set forth in SEQ ID NO: 5; a digestion step in which the DNA is digested with restriction enzyme PacI; and a judging step in which the variety is judged to have low cohumulone ratio when the DNA is not cleaved with restriction enzyme PacI in the digestion step above.

Methods utilizing PCR-RFLP may be mentioned as examples of such a screening method. Specifically, such methods compare the sizes of DNA fragments produced after treatment with a restriction enzyme, based on mutations or nucleotide insertions or deletions at a restriction enzyme recognition site in the genomic DNA or a DNA fragment amplified by PCR using cDNA as template, to determine whether or not a certain trait is present.

The primer used in the PCR step may be synthesized with a DNA synthesizer, selecting a nucleotide sequence of 18-50 contiguous nucleotides from the nucleotide sequence as set forth in SEQ ID NO: 5. The size of the primer is preferably at least 18 and no greater than 30 nucleotides, and more preferably at least 20 and no greater than 22 nucleotides.

In the judging step, DNA fragments from a hop variety specimen digested with restriction enzyme PacI may be fractionated by agarose electrophoresis or polyacrylamide electrophoresis together with a DNA size marker, thus allowing calculation of the sizes of the digested DNA fragments from hop variety specimens based on the relationship between migration distance of the DNA size marker from the well and the sizes of the DNA size marker. If the sizes of the DNA fragments from the hop variety specimen are known, then it is possible to easily judge whether or not the synthesized DNA in the PCR step was cleaved by restriction enzyme PacI, so that the hop variety specimen can be judged as a variety with low cohumulone ratio when cleavage has been confirmed. The digested DNA fragments and size marker in the gel after electrophoresis may be detected under UV irradiation after treatment with, for example, ethidium bromide or SYBR Green.

Furthermore, the screening method of the present invention is a method for screening of hop varieties with low ratio of cohumulone contents relative to α acid contents in hops (cohumulone ratio) which comprises: an extraction step in which genomic DNA is extracted from a hop variety specimen; and an identifying step in which genomic DNA polymorphism at the 1820th nucleotide position of the nucleotide sequence as set forth in SEQ ID NO: 5 is identified.

It is preferred that the identifying step comprises: a PCR step in which DNA is synthesized by PCR using the aforementioned genomic DNA as template, a primer consisting of a nucleotide sequence of 18-50 contiguous nucleotides located toward the 5′-end from the 1820th nucleotide of the nucleotide sequence as set forth in SEQ ID NO: 5 and a primer consisting of the sequence complementary to a nucleotide sequence of 18-50 contiguous nucleotides located toward the 3′-end from the 1820th nucleotide of the nucleotide sequence as set forth in SEQ ID NO: 5; a digestion step in which the DNA is digested with restriction enzyme HpaI; and a judging step in which the variety is judged to have low cohumulone ratio when all kinds of the DNA are cleaved with restriction enzyme HpaI in the digestion step above.

As such a method, for example, a PCR-RFLP method may be mentioned. The specific procedure of the method and synthesis of primers are as mentioned above. The size of primers are preferably not less than 18 and not more than 30 bases, and further preferably not less than 20 and not more than 22 bases.

In the judging step, DNA fragments from a hop variety specimen digested with restriction enzyme HpaI may be fractionated by agarose electrophoresis or polyacrylamide electrophoresis together with a DNA size marker, thus allowing calculation of the sizes of the digested DNA fragments from hop variety specimens based on the relationship between migration distance of the DNA size marker from the well and the sizes of the DNA size marker. If the sizes of the DNA fragments from the hop variety specimen are known, then it is possible to easily judge whether or not the synthesized DNA in the PCR step was cleaved by restriction enzyme HpaI, so that the hop variety specimen can be judged as a variety with low cohumulone ratio when cleavage has been confirmed. The digested DNA fragments and size marker in the gel after electrophoresis may be detected under UV irradiation after treatment with, for example, ethidium bromide or SYBR Green.

It is preferred that the identifying step comprises: a PCR step in which DNA is synthesized by PCR using the aforementioned genomic DNA as template, a primer including the nucleotide sequence as set forth in SEQ ID NO: 7 and a primer including the nucleotide sequence as set forth in SEQ ID NO: 8; a digestion step in which the DNA is digested with restriction enzyme HpaI; and a judging step in which the variety is judged to have low cohumulone ratio when a DNA fragment of about 1304 by is not contained in the DNA fragments obtained by the digestion step above.

The primers to be used in the aforementioned PCR step can be synthesized by a DNA synthesizer separately based on the nucleotide sequence as set forth in SEQ ID NO: 7 and the nucleotide sequence as set forth in SEQ ID NO: 8.

In the judging step, DNA fragments from a hop variety specimen digested with restriction enzyme HpaI may be fractionated by agarose electrophoresis or polyacrylamide electrophoresis together with a DNA size marker, thus allowing calculation of the sizes of the digested DNA fragments from hop variety specimens based on the relationship between migration distance of the DNA size marker from the well and the sizes of the DNA size marker. As a result, if the size of at least one type of DNA fragment derived from the hop variety specimen is not detected in the proximity of a size of 1304 bp, the hop variety specimen can be judged as a variety having low cohumulone ratio. The digested DNA fragments and size marker in the gel after electrophoresis may be detected under UV irradiation after treatment with, for example, ethidium bromide or SYBR Green.

The screening method of the invention may be used for screening of hop varieties with low cohumulone ratio, but it is preferably used for screening of hop varieties with low colupulone ratio, and hop varieties with high contents of farnesene and/or linalool, as well as hop varieties with low cohumulone ratio.

The primer set of the present invention is a primer set to be used for screening of hop varieties with low ratio of cohumulone contents relative to α acid contents in hops, the primer set comprising: a primer containing the nucleotide sequence as set forth in SEQ ID NO: 7; And a primer containing the nucleotide sequence as set forth in SEQ ID NO: 8.

The primer set may be synthesized with a DNA synthesizer based on the nucleotide sequence as set forth in SEQ ID NO: 7 and the nucleotide sequence as set forth in SEQ ID NO: 8.

### Examples

The present invention will now be explained in greater detail with reference to examples, with the understanding that the invention is not meant to be limited to these examples.

1. Experiment Method

### 1) Quantitation of β Acids in Hop Cones:

The quantities of β acids in the hop cones were measured by a modified mode of the official method of the American Society of Brewing Chemists (ASBC). Specifically, 1 g of pulverized hop cones was first placed in a 100 mL stoppered Erlenmeyer flask, and then 4 mL of methanol and 20 mL of diethyl ether were added prior to shaking for 30 minutes. Next, 8 mL of 0.1 M hydrochloric acid was added prior to shaking for 10 minutes, and after standing, 0.2 mL of the ether layer was sampled, methanol was added to a volume of 10 mL and a portion was analyzed by high performance liquid chromatography (HPLC). The HPLC conditions were based on Official Method 7.7 of the European Brewery Convention (EBC), and the α acids, β acids were detected and quantified at a wavelength of 314 nm.

The cohumulone ratio and colupulone ratio were calculated in comparison with the amounts of humulone, cohumulone, lupulone and colupulone of the reference sample of a bittering agent of hop, namely, ICE-2 (manufactured by Labor Veritas AG).

2) Quantitation of Farnesene and Linalool in Hop Cones:

The quantity of farnesene and linalool in hop cones was determined by the following procedure. First, 1 g of pulverized hop cones was placed in a 10 mL stoppered centrifuge tube, and then 6 mL of n-hexane was added prior to shaking for 45 minutes, after which the mixture was centrifuged at 3,000 rpm, 20° C. for 10 minutes. Next, 1.5 mL of the supernatant was sampled in a 10 mL stoppered centrifuge tube, and 3 mL of 5% potassium carbonate was added prior to shaking for 5 minutes, after which the mixture was centrifuged at 3,000 rpm, 20° C. for 10 minutes and the supernatant was analyzed by gas chromatography (HP5890; manufactured by Hewlett-Packard Company). As the column, a J&W capillary column (DB-1, 0.25 mm×30 cm×1.0 μm; manufactured by GL Sciences Inc.) was used. As reference samples for farnesene and linalool, (E)-β-farnesene (manufactured by Wako Pure Chemical Industries Ltd.) and (±)-Linalool (manufactured by SIGMA) were used.

3) Preparation of Total RNA and Poly A⁺ RNA from Lupulin Gland of Hop Cones:

First, the frozen cones of female hop plants were crushed and the fraction that passed through a 250 μm mesh diameter sieve was used as the lupulin gland fraction. The lupulin gland fraction was then homogenized in liquid nitrogen, a 2% CTAB solution (2% cetyltrimethylammonium bromide, 20 mM EDTA, 1.4 M NaCl, 5% β-mercaptoethanol, 0.1 M Tris, pH 9.5) was added to create a suspension, and after warming at 65° C. for 10 minutes, extraction was performed twice with chloroform/isoamyl alcohol (24:1). A ⅓-fold amount of 10 M lithium chloride was added to the obtained water-soluble fraction, and the mixture was allowed to stand overnight and then centrifuged at 15,000 rpm for 10 minutes, after which the obtained precipitate was dissolved in RNase-free sterilized water. Next, a ⅓-fold amount of 10 M lithium chloride was further added to the resulting solution, and the mixture was allowed to stand overnight and then centrifuged at 15,000 rpm for 10 minutes, after which the obtained precipitate was dissolved in RNase-free sterilized water for use as the total RNA sample.

The obtained total RNA sample was quantitated by measurement of the absorbance at 260 nm using a spectrophotometer, and then Oligotex-dT30 Super by Takara Bio, Inc. was used for preparation of poly A⁺ RNA following the manufacturer's protocol.

4) Preparation of Hop Cone Lupulin Gland cDNA Library:

A Creator SMART cDNA Library Construction Kit by BD Biosciences was used to prepare a hop cone lupulin gland cDNA library from the poly A⁺ RNA obtained from hop cone lupulin gland. The procedure for preparation of the cDNA library was according to the manufacturer's protocol supplied with the kit.

2. Experimental Results

### Example 1

Search for breeding markers related to cohumulone ratio, colupulone ratio, farnesene contents and linalool contents.

First, clones were randomly selected from a hop cone (Saaz variety) lupulin gland cDNA library, and lupulin gland cDNA introduced into each clone was used as template for PCR in the presence of digoxigenin (DIG)-labeled dUTP, to create DIG-labeled probes. The DIG-labeled probes were created by PCR using a PCR DIG probe synthesis kit by Roche Diagnostics K.K.

Next, genomic DNA was extracted from 110 hybrid progeny of Chinook and SaM hop varieties stocked by Sapporo Breweries Ltd. and analyzed by RFLP using combinations of the aforementioned DIG-labeled probes and restriction enzymes EcoT22I or XbaI, and a search was conducted for breeding markers that can detect polymorphisms correlated with cohumulone ratio, colupulone ratio, farnesene contents and linalool contents. The genomic DNA extraction and genomic Southern hybridization were conducted according to the method described in Molecular cloning (Maniatis et al., 1989, Cold Spring Harbor Laboratory Press).

As a result, polymorphism detected with the combination of the cDNA clone 3D03 (hereinafter “3D03”) from lupulin gland and restriction enzyme XbaI (hereinafter, “XbaI/3D03 polymorphism”) was shown to be correlated with cohumulone ratio, colupulone ratio, farnesene contents and linalool contents, suggesting that it can be utilized as a breeding marker.

In Example 2 it was examined whether 3D03 can be utilized as a breeding marker correlated with cohumulone ratio, colupulone ratio, farnesene contents and linalool contents in a wide range of hop lines other than the hybrid progeny of the Chinook and SaM varieties. The nucleotide sequence of 3D03 is listed as SEQ ID NO: 1.

### Example 2

Correlation between cohumulone ratio, colupulone ratio, farnesene contents or linalool contents and XbaI/3D03 polymorphism:

Nine hop lines stocked by Sapporo Breweries Ltd. (Daimanshu, 980690, M916001003, Hokusenzairai, 0004B, 971174, 980573, Monica, M843501246) were selected and crossbred to obtain hybrid progeny, the cohumulone ratio, colupulone ratio, farnesene contents and linalool contents of the hop cones were examined, and their correlation with XbaI/3D03 polymorphism was determined.

The cross breeding was carried out with the combinations Daimanshu×980690, Daimanshu×M916001003, Hokusenzairai×980690, 0004B×971174, 0004B×980573, Monica×M843501246, Monica×980573, and the cohumulone ratio, colupulone ratio, farnesene contents and linalool contents in the cones of the obtained hybrid progeny were examined.

Correlation between the XbaI/3D03 polymorphism and cohumulone ratio, colupulone ratio, farnesene contents and linalool contents was judged by dividing the hybrid progeny obtained by cross breeding into groups based on XbaI/3D03 polymorphism, calculating the mean values for the component contents in each group, and determining statistically significant difference by T test. For the T test, the difference in variance of measured values among the XbaI/3D03 polymorphisms was investigated by F test for each hop line combination, and homoskedasticity was assumed when no difference in variance was found, while variance was assumed to be unequal when a difference in variance was found. The contribution ratio representing the effect of XbaI/3D03 polymorphisms on cohumulone ratio, colupulone ratio, farnesene contents and linalool contents was calculated as genetic variance/population variance. The genetic variance was the dispersion for a theoretical population experiencing no factors other than the subject genotype 3D03 (no influence by other gene loci or environment). Specifically, if the mean value for the parent population is represented as XP, the population variance as VP, the mean value for a population with genotypes 1, 2, . . . as X1, X2, . . . and the number of individuals in each genotype as N1, N2, then the genetic variance (V_{G}) and contribution ratio (H) may be calculated by the following formulas.

Genetic variance (V_{G})=((X_{P}−X₁)²×N₁+(X_{P}−X₂)²×N₂+ . . . )/(N₁+N₂+ . . . )

Contribution ratio (H)=V_{G}/V_{P}

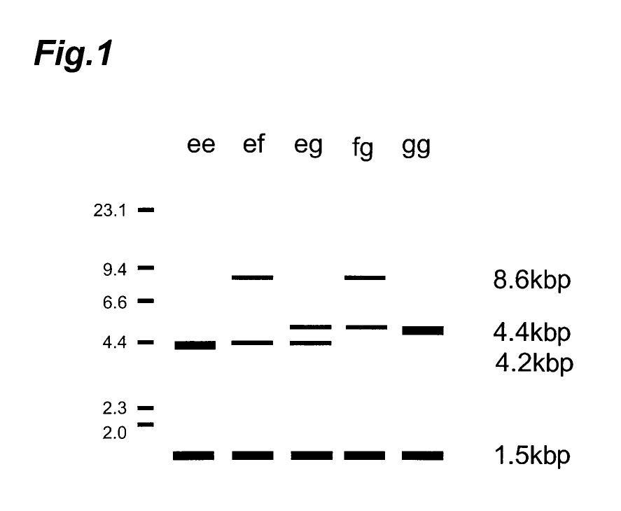

FIG. 1 is an image showing XbaI/3D03 polymorphism, obtained by digestion of genomic DNA of different hop varieties with restriction enzyme XbaI, and genomic Southern hybridization. The hybrid progeny obtained by the different cross breeding combinations exhibited five different patterns, ee-type, ef-type, eg-type, fg-type and gg-type.

Table 1 shows the correlation between XbaI/3D03 polymorphism and cohumulone ratio.

TABLE 1

| | ee | | | ef | | | eg | | |
|---|---|---|---|---|---|---|---|---|---|
| | Average | Significant | Size of | Average | Significant | Size of | Average | Significant | Size of |
| | value (%) | difference | population | value (%) | difference | population | value (%) | difference | population |
| Daimanshu × | 22.9 | a | 7 | 36.6 | ab | 2 | 24.4 | a | 12 |
| 980690 | | | | | | | | | |
| Daimanshu × | | | | | | | 22.7 | a | 17 |
| M916001003 | | | | | | | | | |
| Hokusenzairai × | 29.2 | ab | 6 | 36.3 | ab | 5 | 26.2 | a | 9 |
| 980690 | | | | | | | | | |
| 0004B × 971174 | | | | | | | | | |
| 0004B × 980573 | | | | | | | | | |
| Monica × | 31.6 | N.S. | 9 | | | | 29.8 | N.S. | 27 |
| M843501246 | | | | | | | | | |
| Monica × 980573 | | | | 38.7 | b | 12 | 27.7 | a | 13 |

| | fg | | | gg | | | Contri- |
|---|---|---|---|---|---|---|---|
| | Average | Significant | Size of | Average | Significant | Size of | bution |
| | value (%) | difference | population | value (%) | difference | population | ratio |
| Daimanshu × | 34.7 | b | 9 | | | | 61.49 |
| 980690 | | | | | | | |
| Daimanshu × | 33.6 | b | 8 | | | | 64.38 |
| M916001003 | | | | | | | |
| Hokusenzairai × | 39.0 | b | 13 | | | | 49.10 |
| 980690 | | | | | | | |
| 0004B × 971174 | 36.2 | a | 9 | 26.8 | b | 28 | 45.41 |
| 0004B × 980573 | 41.3 | a | 18 | 26.8 | b | 18 | 67.57 |
| Monica × | | | | 28.3 | N.S. | 3 | 3.75 |
| M843501246 | | | | | | | |
| Monica × 980573 | 39.8 | b | 18 | 31.0 | ab | 7 | 38.22 |

The significant difference is shown at a significance level of 5% or less. The difference between a and b is significant; whereas, the differences between a and ab, and ab and b are not significant. N.S. represents no significant difference.

As a result, it was observed that the hybrid progeny represented by of type and fg type tend to have high cohumulone ratio; whereas, the hybrid progeny represented by ee type, eg type and gg type tend to have low cohumulone ratio. These results suggested that a hop variety without an f-type genome has a lower cohumulone ratio than a hop variety containing at least one f-type genome.

Table 2 shows the correlation between XbaI/3D03 polymorphism and colupulone ratio.

TABLE 2

| | ee | | | ef | | | eg | | |
|---|---|---|---|---|---|---|---|---|---|
| | Average | Significant | Size of | Average | Significant | Size of | Average | Significant | Size of |
| | value (%) | difference | population | value (%) | difference | population | value (%) | difference | population |
| Daimanshu × | 44.0 | a | 7 | 61.7 | ab | 2 | 46.2 | a | 12 |
| 980690 | | | | | | | | | |
| Daimanshu × | | | | | | | 46.5 | a | 17 |
| M916001003 | | | | | | | | | |
| Hokusenzairai × | 49.0 | ab | 6 | 62.0 | b | 5 | 47.1 | a | 9 |
| 980690 | | | | | | | | | |
| 0004B × 971174 | | | | | | | | | |
| 0004B × 980573 | | | | | | | | | |
| Monica × | 58.5 | N.S. | 9 | | | | 57.0 | N.S. | 27 |
| M843501246 | | | | | | | | | |
| Monica × 980573 | | | | 65.8 | b | 12 | 55.3 | a | 13 |

| | fg | | | gg | | | Contri- |
|---|---|---|---|---|---|---|---|
| | Average | Significant | Size of | Average | Significant | Size of | bution |
| | value (%) | difference | population | value (%) | difference | population | ratio |
| Daimanshu × | 59.5 | b | 9 | | | | 74.65 |
| 980690 | | | | | | | |
| Daimanshu × | 57.6 | b | 8 | | | | 58.16 |
| M916001003 | | | | | | | |
| Hokusenzairai × | 63.4 | bc | 13 | | | | 63.99 |
| 980690 | | | | | | | |
| 0004B × 971174 | 58.5 | a | 9 | 48.5 | b | 28 | 48.46 |
| 0004B × 980573 | 61.1 | a | 18 | 47.3 | b | 18 | 66.47 |
| Monica × | | | | 55.0 | N.S. | 3 | 3.69 |
| M843501246 | | | | | | | |
| Monica × 980573 | 65.4 | b | 18 | 56.2 | ab | 7 | 39.81 |

The significant difference is shown at a significance level of 5% or less. The difference between a and b is significant; whereas, the differences between a and ab, and ab and b are not significant. N.S. represents no significant difference.

As a result, it was observed that the hybrid progeny represented by of type and fg type tend to have high colupulone ratio; whereas, the hybrid progeny represented by ee type, eg type and gg type tend to have low colupulone ratio. These results suggested that a hop variety without an f-type genome has a lower colupulone ratio than a hop variety containing at least one f-type genome.

Table 3 shows the correlation between XbaI/3D03 polymorphism and farnesene contents.

TABLE 3

| | ee | | | ef | | | eg | | |
|---|---|---|---|---|---|---|---|---|---|
| | Average | | | Average | | | Average | | |
| | value (mg/ | Significant | Size of | value (mg/ | Significant | Size of | value (mg/ | Significant | Size of |
| | g cone) | difference | population | g cone) | difference | population | g cone) | difference | population |
| Daimanshu × | 0.02 | N.S. | 7 | 0.01 | N.S. | 2 | 0.02 | N.S. | 12 |
| 980690 | | | | | | | | | |
| Daimanshu × | | | | | | | 0.49 | a | 17 |
| M916001003 | | | | | | | | | |
| Hokusenzairai × | 0.64 | N.S. | 6 | 0.30 | N.S. | 5 | 0.52 | N.S. | 9 |
| 980690 | | | | | | | | | |
| 0004B × 971174 | | | | | | | | | |
| 0004B × 980573 | | | | | | | | | |
| Monica × | 2.84 | N.S. | 9 | | | | 3.56 | N.S. | 27 |
| M843501246 | | | | | | | | | |
| Monica × 980573 | | | | 2.24 | N.S. | 12 | 4.32 | N.S. | 13 |

| | fg | | | gg | | | |
|---|---|---|---|---|---|---|---|
| | Average | | | Average | | | Contri- |
| | value (mg/ | Significant | Size of | value (mg/ | Significant | Size of | bution |
| | g cone) | difference | population | g cone) | difference | population | ratio |
| Daimanshu × | 0.02 | N.S. | 9 | | | | 2.63 |
| 980690 | | | | | | | |
| Daimanshu × | 0.24 | b | 8 | | | | 12.88 |
| M916001003 | | | | | | | |
| Hokusenzairai × | 0.35 | N.S. | 13 | | | | 15.92 |
| 980690 | | | | | | | |
| 0004B × 971174 | 1.68 | a | 9 | 2.84 | b | 28 | 14.42 |
| 0004B × 980573 | 2.50 | N.S. | 18 | 2.35 | N.S. | 18 | 0.24 |
| Monica × | | | | 3.38 | N.S. | 3 | 2.65 |
| M843501246 | | | | | | | |
| Monica × 980573 | 2.69 | N.S. | 18 | 3.15 | N.S. | 7 | 28.12 |

The significant difference is shown at a significance level of 5% or less. The difference between a and b is significant; whereas, the differences between a and ab, and ab and b are not significant. N.S. represents no significant difference.

As a result, it was observed that the hybrid progeny represented by of type and fg type tend to have low farnesene contents; whereas, the hybrid progeny represented by ee type, eg type and gg type tend to have high farnesene contents. These results suggested that a hop variety without an f-type genome has a higher farnesene contents than a hop variety containing at least one f-type genome.

Table 4 shows the correlation between XbaI/3D03 polymorphism and linalool contents.

TABLE 4

| | ee | | | ef | | | eg | | |
|---|---|---|---|---|---|---|---|---|---|
| | Average | | | Average | | | Average | | |
| | value (mg/ | Significant | Size of | value (mg/ | Significant | Size of | value (mg/ | Significant | Size of |
| | g cone) | difference | population | g cone) | difference | population | g cone) | difference | population |
| Daimanshu × | 0.03 | N.S. | 7 | 0.08 | N.S. | 2 | 0.04 | N.S. | 12 |
| 980690 | | | | | | | | | |
| Daimanshu × | | | | | | | 0.38 | a | 17 |
| M916001003 | | | | | | | | | |
| Hokusenzairai × | 0.46 | N.S. | 6 | 0.28 | N.S. | 5 | 0.36 | N.S. | 9 |
| 980690 | | | | | | | | | |
| 0004B × 971174 | | | | | | | | | |
| 0004B × 980573 | | | | | | | | | |
| Monica × | 1.68 | N.S. | 9 | | | | 2.21 | N.S. | 27 |
| M843501246 | | | | | | | | | |
| Monica × 980573 | | | | 1.35 | N.S. | 12 | 2.24 | N.S. | 13 |

| | fg | | | gg | | | |
|---|---|---|---|---|---|---|---|
| | Average | | | Average | | | Contri- |
| | value (mg/ | Significant | Size of | value (mg/ | Significant | Size of | bution |
| | g cone)) | difference | population | g cone) | difference | population | ratio |
| Daimanshu × | 0.04 | N.S. | 9 | | | | 15.17 |
| 980690 | | | | | | | |
| Daimanshu × | 0.21 | b | 8 | | | | 11.68 |
| M916001003 | | | | | | | |
| Hokusenzairai × | 0.28 | N.S. | 13 | | | | 10.87 |
| 980690 | | | | | | | |
| 0004B × 971174 | 1.05 | a | 9 | 1.58 | b | 28 | 11.92 |
| 0004B × 980573 | 1.64 | N.S. | 18 | 1.90 | N.S. | 18 | 1.62 |
| Monica × | | | | 2.68 | N.S. | 3 | 7.59 |
| M843501246 | | | | | | | |
| Monica × 980573 | 1.41 | N.S. | 18 | 1.63 | N.S. | 7 | 27.48 |

The significant difference is shown at a significance level of 5% or less. The difference between a and b is significant; whereas, the differences between a and ab, and ab and b are not significant. N.S. represents no significant difference.

As a result, it was observed that the hybrid progeny represented by of type and fg type tend to have low linalool contents; whereas, the cross progenies of ee type, eg type and gg type tend to have high linalool contents. These results suggested that a hop variety without an f-type genome has a higher linalool contents than a hop variety containing at least one f-type genome.

The results mentioned above suggest that whether cohumulone ratio, colupulone ratio, farnesene contents and linalool contents are high or low can be estimated by analyzing an arbitrarily chosen hop variety for an XbaI/3D03 polymorphism, and that a hop variety without an f-type genome is one having low cohumulone ratio and colupulone ratio, and high farnesene contents and linalool contents.

### Example 4

### Cloning of Genomic DNA Fragment Containing 3D03

Since it was suggested that a hop variety without f-type genome has low cohumulone ratio and colupulone ratio, and high farnesene contents and linalool contents, the nucleotide sequence of 3D03 was used for cloning of e-type, f-type and g-type genomic DNA fragments, and difference of nucleotide sequences thereof was analized. In this manner, identification of polymorphism at 3D03 gene locus was attempted.

First, the following primers were designed based on the sequences at both end regions of 3D03.

p3D03-F1 primer:

| | |
|---|---|
| 5′-ACTAGTTTCTCTCCACAATCTCTGC-3′ | (SEQ ID NO: 2) |
| p3D03-R1 primer: | |
| 5′-GGTGGAATATCATTATTGTGTCAGC-3′ | (SEQ ID NO: 3) |

Next, genomic DNA was extracted from Chinook varieties carrying hetero genomes of e-type and f-type and a SaM variety carrying homologous genomes of g-type, PCR was conducted using the p3D03-F1 primer and p3D03-R1 primer, and the specifically amplified genomic DNA fragments were cloned. The nucleotide sequences were analyzed for the clones of the DNA fragments cloned from the Chinook variety genomic DNA and the clones of the DNA fragments cloned from the SaM variety genomic DNA. The PCR was conducted using PrimeSTAR HS DNA Polymerase by Takara Bio, Inc., according to the manufacturer's protocol. The nucleotide sequence analysis was carried out utilizing the Macrogen Data Analysis Service.

As a result, the DNA fragments cloned from the genomic DNA of breed Chinook were classified into two types of nucleotide sequences. One of them was cleaved by restriction enzyme HpaI, whereas the other one was not cleaved by restriction enzyme HpaI. Using the genomic DNA of breed Nugget having homologous genomes of e-type as a template, PCR was performed using p3D03-F1 primer and p3D03-R1 primer and whether the specifically amplified DNA fragments were cleaved by restriction enzyme HpaI or not was checked. As a result, it was found that the DNA fragment derived from the e-type genome is cleaved by restriction enzyme HpaI. Of the DNA fragments cloned from the genomic DNA of breed Chinook, the nucleotide sequences cleaved by restriction enzyme HpaI were identified as e-type and the nucleotide sequences not cleaved by restriction enzyme HpaI were identified as f-type. Furthermore, since breed SaM has homologous genomes of g type, the nucleotide sequences of the DNA fragments cloned form the genomic DNA of breed SaM were identified as g-type. The nucleotide sequence of e-type genomic DNA fragment (e-type g3DO3) is shown in SEQ ID NO: 4 of the sequence listing. The nucleotide sequence of f-type genomic DNA fragment (f-type g3DO3) is shown in SEQ ID NO: 5. The nucleotide sequence of g-type genomic DNA fragment (g-type g3DO3) is shown in SEQ ID No: 6.

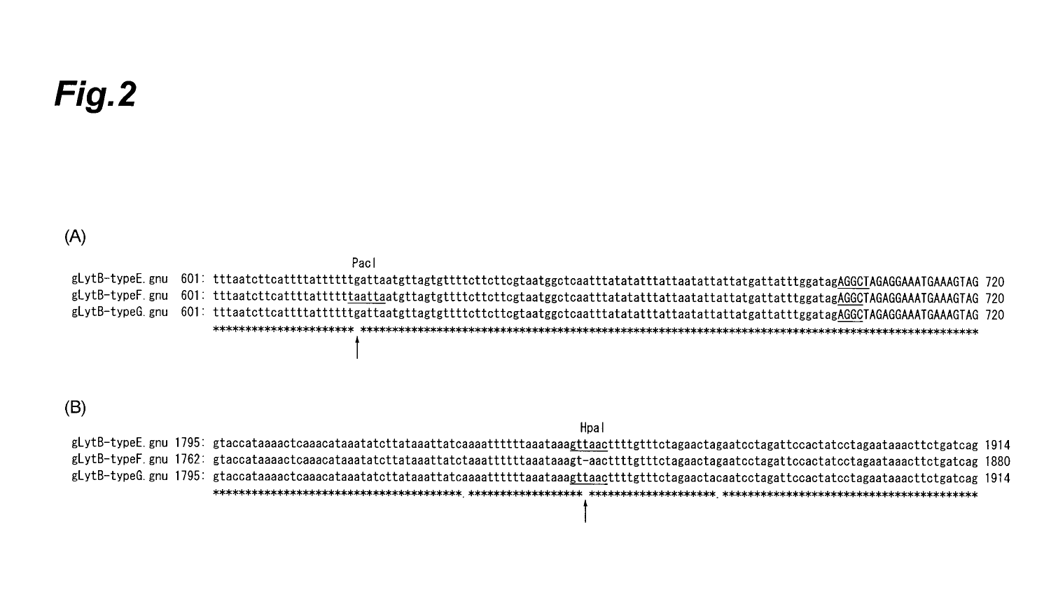

FIG. 2 shows homology of the nucleotide sequences of e-type g3DO3, f-type g3DO3 and g-type g3DO3. According to the results, a single nucleotide polymorphism was found at the 623rd nucleotide of f-type g3DO3 indicated by the arrow in FIG. 2(A), and at the 1820th nucleotide of f-type g3DO3 indicated by the arrow in FIG. 2(B). It was demonstrated that a single nucleotide polymorphism at the 623rd nucleotide of f-type g3DO3 can be distinguished from e-type g3DO3 and g-type g3DO3 by cleavage with restriction enzyme PacI, and a single nucleotide polymorphism at the 1820th nucleotide of f-type g3DO3 can be distinguished from e-type g3DO3 and g-type g3DO3 by no cleavage with restriction enzyme HpaI.

### Example 5

### Analysis of Single Nucleotide Polymorphism Found in f-Type g3DO3

Since it had been determined that f-type g3DO3 contains a single nucleotide polymorphism that can be distinguished by no cleavage with restriction enzyme HpaI, it was then investigated whether this single nucleotide polymorphism can be utilized to screen for hop varieties with low cohumulone ratio and colupulone ratio, and high farnesene contents and linalool contents, in the same manner as the XbaI/3D03 polymorphism.

First, the genomic DNA of 9 hop lines (Daimanshu, 980690, M916001003, Hokusenzairai, 0004B, 971174, 980573, Monica, M843501246) were used for PCR-RFLP analysis using a combination of restriction enzyme HpaI and the following primer set consisting of p3DO3-F2 primer and p3DO3-R2 primer, and the resulting polymorphism (hereinafter referred to as “HpaI/g3D03 polymorphism”) was detected.

| p3D03-F2 primer: | |
|---|---|
| 5′-TGCTGTTTCTAAGGGATTCG-3′ | (SEQ ID NO: 7) |
| p3D03-R2 primer: | |
| 5′-TTTCCAGGACCTATTCTCTG-3′ | (SEQ ID NO: 8) |

Specifically, DNA was synthesized by PCR using the genomic DNA of the 9 hop lines as template with p3D03-F2 primer and p3D03-R2 primer, and then the DNA was digested with restriction enzyme HpaI, the digested DNA fragments were fractionated by 1% agarose gel electrophoresis, and the band pattern of the fractionated DNA fragments in the agarose gel was examined. Detection of an approximately 1304 by band in analysis of the HpaI/g3D03 polymorphism means that the genomic DNA has the f-type genome, while detection of an approximately 1138 by band means that the genomic DNA has the 1-type genome.

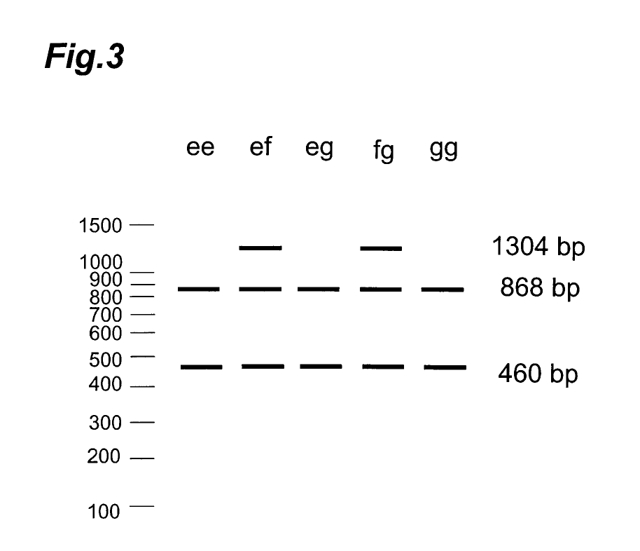

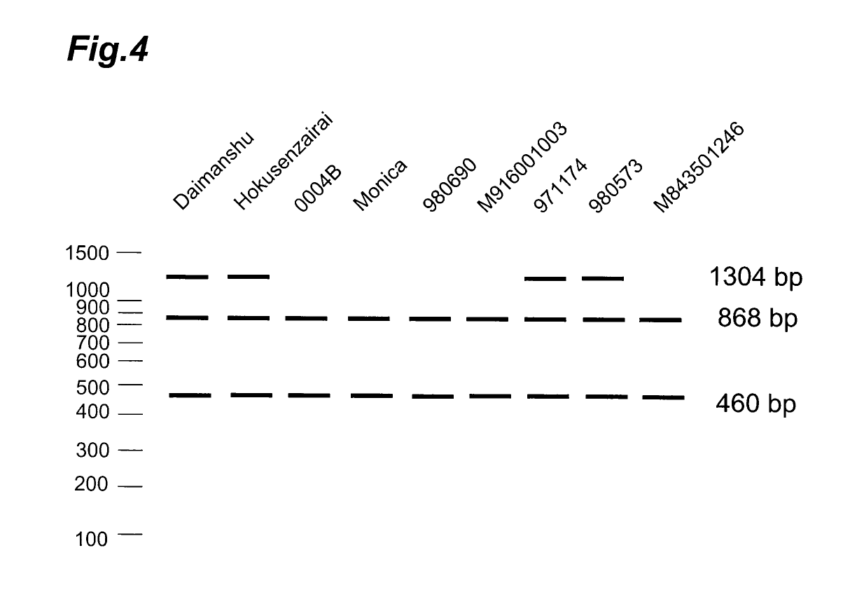

FIG. 3 shows a typical band pattern for HpaI/g3D03 polymorphism, obtained by PCR-RFLP analysis of hop varieties, and FIG. 4 shows HpaI/g3D03 polymorphism obtained by analysis of the genomic DNA of the 9 hop lines.

The results suggested that Daimanshu, Hokusenzairai, 971174 and 980573 are hop lines with the f-type genome, 0004B, Monica, 980690, M916001003 and M843501246 are hop lines without the f-type genome.

Next, genomic DNA from the same 9 hop lines was used for RFLP analysis with a combination of restriction enzyme XbaI and 3D03, and the obtained XbaI/g3D03 polymorphisms were examined to confirm whether or not the determined results based on HpaI/g3D03 polymorphism were correct.

Specifically, the genomic DNA of each of the 9 hop lines was cleaved with restriction enzyme XbaI, the digested genomic DNA fragments were fractionated by 1% agarose gel electrophoresis, the fractionated genomic DNA fragments were transferred to a nylon membrane, labeled 3D03 was used as a probe for hybridization, and the hybridized bands were detected based on the probe labeling.

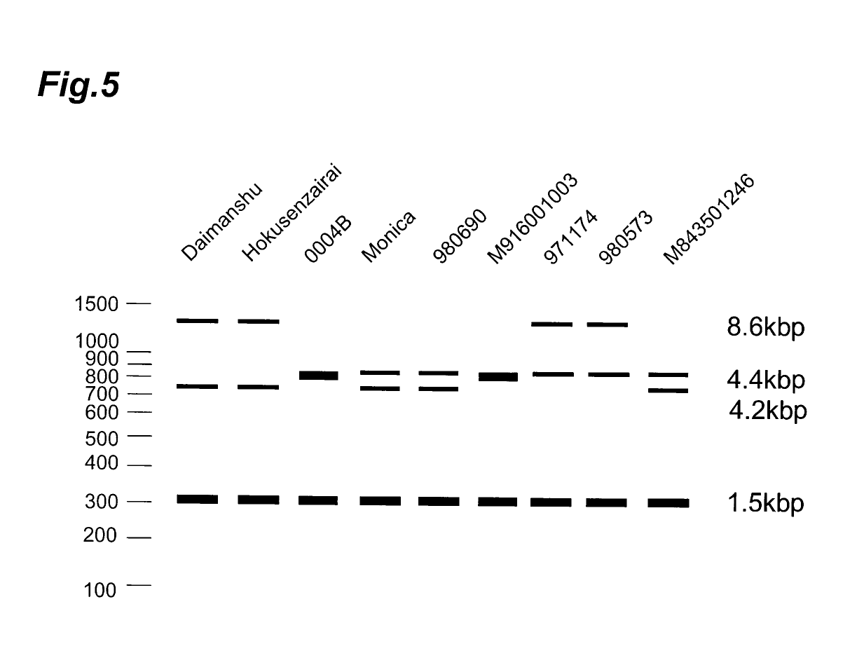

FIG. 5 is an image showing XbaI/g3D03 polymorphism, obtained by genomic analysis of the 9 hop lines.

The results suggested that Daimanshu, Hokusenzairai, 971174 and 980573 are hop lines with the f-type genome, 0004B, Monica, 980690, M916001003 and M843501246 are hop lines without the f-type genome.

The results described above demonstrated that analysis of HpaI/g3D03 polymorphism, like analysis of XbaI/g3D03 polymorphism, can determine whether or not a hop variety has the f-type genome, thus suggesting that it can be used as a screening method for hop varieties with low cohumulone ratio and colupulone ratio, and high farnesene contents and linalool contents.

### INDUSTRIAL APPLICABILITY

According to the invention it is possible to provide a breeding marker for hop varieties with low cohumulone ratio, the breeding marker also being usable as a breeding marker for hop varieties with low colupulone ratio, and for hop varieties with high farnesene contents and/or linalool contents. According to the invention it is also possible to objectively and rapidly screen for hop varieties with low cohumulone ratio and colupulone ratio, and high farnesene contents and linalool contents.

1. A breeding marker represented by any one of the following (a) to (d), which is used for screening of hop varieties with low ratio of cohumulone contents relative to α acid contents in hops.
(a) A polynucleotide consisting of a nucleotide sequence of 20-3434 continuous nucleotides including nucleotide No.623 of the polynucleotide consisting of the nucleotide sequence as set forth in SEQ ID NO: 5 (wherein the 623th nucleotide has adenine).
(b) A polynucleotide consisting of the sequence complementary to the polynucleotide of (a) above.
(c) A polynucleotide consisting of a nucleotide sequence of 20-3434 continuous nucleotides including nucleotide No.1820 of the polynucleotide consisting of the nucleotide sequence as set forth in SEQ ID NO: 5 (wherein the 1820th nucleotide has adenine).
(d) A polynucleotide consisting of the sequence complementary to the polynucleotide of (c) above.

2. A breeding marker according to claim 1, which is used for screening of hop varieties with low ratio of colupulone contents relative to β acid contents in hops.

3. A breeding marker according to claim 1 or 2, which is used for screening of hop varieties with high contents of farnesene and/or linalool.

4. A screening method for hop varieties with low ratio of cohumulone contents relative to α acid contents in hops, comprising
an extraction step in which genomic DNA is extracted from a hop variety specimen,
a digestion step in which the genomic DNA is digested with restriction enzyme XbaI to obtain digested genomic DNA fragments,
a detection step in which the digested genomic DNA fragments are separated and genomic DNA fragments that hybridize to any one of the polynucleotide of (a) to (d) above are detected, and
a judging step in which it is judged that the variety has low cohumulone ratio when a genomic DNA fragment of about 8.6 kbp is not detected in the detection step above.

5. A method for screening of hop varieties with low ratio of cohumulone contents relative to α acid contents in hops which comprises
an extraction step in which genomic DNA is extracted from a hop variety specimen, and
an identifying step in which genomic DNA polymorphism at the 623rd nucleotide position of the nucleotide sequence as set forth in SEQ ID NO: 5 is identified.

6. A screening method according to claim 5, wherein the identifying step comprises
a PCR step in which DNA is synthesized by PCR using the aforementioned genomic DNA as template, a primer consisting of a nucleotide sequence of 18-50 contiguous nucleotides located toward the 5′-end from the 623rd nucleotide of the nucleotide sequence as set forth in SEQ ID NO: 5 and a primer consisting of the sequence complementary to a nucleotide sequence of 18-50 contiguous nucleotides located toward the 3′-end from the 623rd nucleotide of the nucleotide sequence as set forth in SEQ ID NO: 5,
a digestion step in which the DNA is digested with restriction enzyme PacI, and
a judging step in which the variety is judged to have low cohumulone ratio when the DNA is not cleaved with restriction enzyme PacI in the digestion step above.

7. A method for screening of hop varieties with low ratio of cohumulone contents relative to α acid contents in hops which comprises
an extraction step in which genomic DNA is extracted from a hop variety specimen, and
an identifying step in which genomic DNA polymorphism at the 1820th nucleotide position of the nucleotide sequence as set forth in SEQ ID NO: 5 is identified.

8. A screening method according to claim 7, wherein the identifying step comprises
a PCR step in which DNA is synthesized by PCR using the aforementioned genomic DNA as template, a primer consisting of a nucleotide sequence of 18-50 contiguous nucleotides located toward the 5′-end from the 1820th nucleotide of the nucleotide sequence as set forth in SEQ ID NO: 5 and a primer consisting of the sequence complementary to a nucleotide sequence of 18-50 contiguous nucleotides located toward the 3′-end from the 1820th nucleotide of the nucleotide sequence as set forth in SEQ ID NO: 5,
a digestion step in which the DNA is digested with restriction enzyme HpaI, and
a judging step in which the variety is judged to have low cohumulone ratio when all kinds of the DNA are cleaved with restriction enzyme HpaI in the digestion step above.

9. A screening method according to claim 7, wherein the identifying step comprises
a PCR step in which DNA is synthesized by PCR using the aforementioned genomic DNA as template, a primer including the nucleotide sequence as set forth in SEQ ID NO: 7 and a primer including the nucleotide sequence as set forth in SEQ ID NO: 8,
a digestion step in which the DNA is digested with restriction enzyme HpaI, and
a judging step in which the variety is judged to have low cohumulone ratio when a DNA fragment of about 1304 by is not contained in the DNA fragments obtained by the digestion step above.

10. A screening method according to any one of claims 4 to 9, which is a method for screening of hop varieties with low ratio of colupulone contents relative to β acid contents in hops.

11. A screening method according to any one of claims 4 to 9, which is a method for screening of hop varieties with high contents of farnesene and/or linalool.

12. A primer set to be used for screening of hop varieties with low ratio of cohumulone contents relative to α acid contents in hops, the primer set comprising
a primer containing the nucleotide sequence as set forth in SEQ ID NO: 7 and
a primer containing the nucleotide sequence as set forth in SEQ ID NO: 8.
